(19) United States
(12) Patent Application Publication
(10) Pub. No.: US 2021/0128349 A1
(43) Pub. Date: May 6, 2021

(54) EYE MISTING DEVICE

(71) Applicant: Mistifeye, Inc., Fairfax, VA (US)

(72) Inventors: Jai Karnik, Madison, WI (US); Reagan Jung, Madison, WI (US)

(21) Appl. No.: 16/676,429

(22) Filed: Nov. 6, 2019

(51) Int. Cl. A61F 9/00

(52) U.S. Cl. CPC A61F 9/0008

(57) ABSTRACT

An eye misting device is described. The device includes a graspable housing and a mist aperture in the graspable housing. The device also includes a pod holder receiver formed integral the housing and a pod holder configured to engage the pod holder receiver. Further, the device includes a pod receiving cavity formed within the pod holder for holding an eye solution pod.

### BACKGROUND

Eye drops which may be medicinal or may just be used for soothing or wetting the eye are conventionally difficult to use for many people. Many people have difficulties in getting the drops into the eye, such as young people, elderly, people with tremors of the hand, etc. Even when people are able to place the drops in the eye, they often find the process uncomfortable and often much of the eye drop runs out of the eye and is wasted.

Accordingly, there is a need for a system which provides an improved experience at delivering eye drops (medicinal or not) to the eye. An improved experience may include ease of use, ability to easily deliver the drop solution to the eye with little ability or skill, and improved comfort, among others. There is also a need for a device which allows a user to not have to forcibly hold the eye open during delivery of the drops. There is also a need for a device which provides ease of use and comfort by the user.

### SUMMARY

An exemplary embodiment relates to an eye misting device. The device includes a graspable housing and a mist aperture in the graspable housing. The device also includes a pod holder receiver formed integral the housing and a pod holder configured to engage the pod holder receiver. Further, the device includes a pod receiving cavity formed within the pod holder for holding an eye solution pod.

Another exemplary embodiment relates to an eye misting device. The eye misting device includes a graspable housing and a mist aperture in the graspable housing. The device also includes a pod holder configured to engage the housing and a pod receiving cavity formed within the pod holder for holding an eye solution pod. Further, the device includes a piezoelectric mesh disk configured to create a mist of eye solution when electrified.

Yet another exemplary embodiment relates to an eye misting device. The device includes a graspable housing and a mist aperture in the graspable housing. The device also includes a battery within the housing and a pod holder configured to engage the housing. Further, the device includes a pod receiving cavity formed within the pod holder for holding an eye solution pod and a piezoelectric mesh disk configured to create a mist of eye solution when electrified by the battery.

In addition to the foregoing, other system aspects are described in the claims, drawings, and text forming a part of the disclosure set forth herein. The foregoing is a summary and thus may contain simplifications, generalizations, inclusions, and/or omissions of detail; consequently, those skilled in the art will appreciate that the summary is illustrative only and is NOT intended to be in any way limiting. Other aspects, features, and advantages of the devices and/or processes and/or other subject matter described herein will become apparent in the disclosures set forth herein.

### BRIEF DESCRIPTION OF THE DRAWINGS

FIG. 1 is a depiction of an exemplary eye misting device.

FIG. 2 is an exemplary embodiment of an eye misting device in use.

FIG. 3 is an exemplary embodiment of a pod holder of an eye misting device.

FIG. 4 is an exemplary embodiment of the pod holder of FIG. 3.

FIG. 5 is an exemplary embodiment of an eye solution pod.

The use of the same symbols in different drawings typically indicates similar or identical items unless context dictates otherwise.

### DETAILED DESCRIPTION

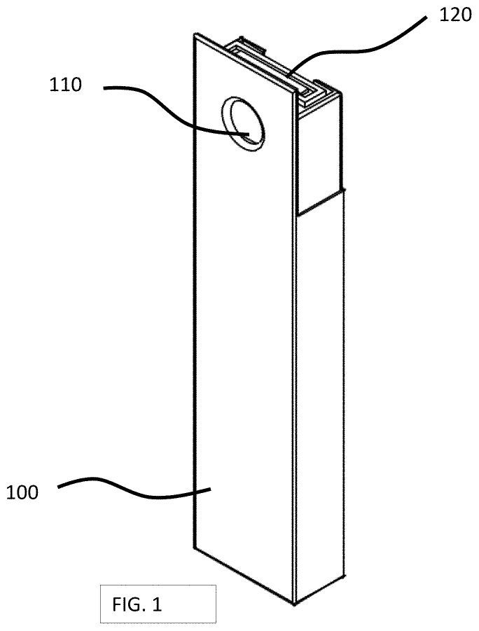

Referring to FIG. 1, in accordance with an exemplary embodiment, an eye misting device 100 uses a piezoelectric mesh disk to convert liquid eye drops into a mist. The mist generated contains, according to one embodiment, tiny micro-droplets sized between 5 um-25 um. The piezo mesh disk vibrates at a resonant frequency between 100 kHz-200 kHz depending on the desired velocity of the mist.

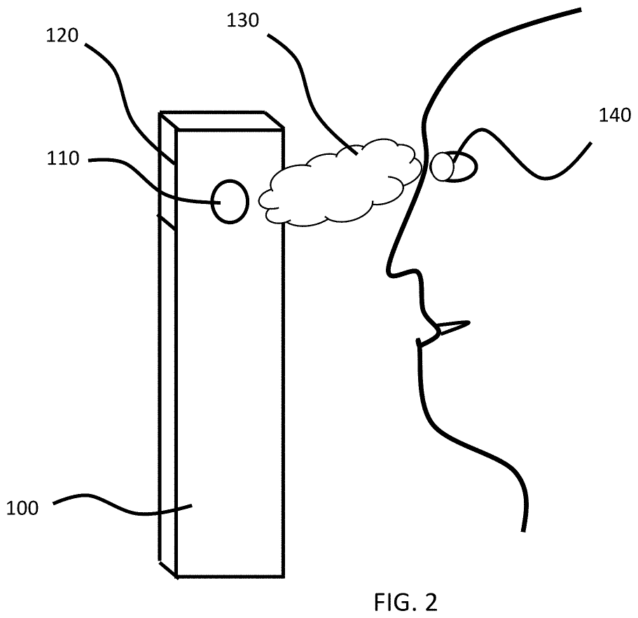

As depicted in FIG. 1, the mist exits device 100 through an aperture 110. A detachable capsule or pod 120 holds the eye drop solution and delivers the solution to the piezoelectric mesh disk. Referring now to FIG. 2, a mist is applied at a comfortable distance (e.g., 2-4 inch) from each eye 140. In accordance with some exemplary embodiments 3 seconds of applying the mist equals to 1 dose of applied medication. One dose approximately equals to anywhere between 7 uL-20 uL of medicine which meets the cornea and is enough to reach therapeutic levels.

In accordance with an exemplary embodiment, a pre-filled disposable pod 120 is configured to hold any of a variety of eye solutions including but not limited to over-the-counter eye drops, medicinal drops, etc. (which would last the user for 3-5 days, e.g.). Pods 120 may be made using recyclable materials, biodegradable materials, or any other type of materials. Pods 120 are inserted into the device before use. Upon insertion, a puncturable seal in pods 120 is punctured releasing the solution. The solution then meets a piezo disk before being converted into a gentle mist. In accordance with exemplary embodiments, device 100 may contain a UV light to sanitize the piezoelectric disk for prolonged use. Device 100 may be configured as a handheld device that is chargeable using a USB or other electrical connector.

In use, device 100 creates a fine mist that is directed at the eye. Users do not need to force their eyes open during delivery, the user can blink naturally. The gentle vapor or mist may be soothing and is configured not to cause any blink-reflex reaction. None of our competitors provide that.

The device is typically used from a comfortable/non-intimidating distance (2-4 inches) from the eye which is easy on the user. Users can position device 100 at any angle making it very easy to use by preventing “missed drops”. Device 100 differs from other eye solution delivery devices which rely on fast spraying technology which needs to be used very close to the eyes and at a precise location.

In accordance with exemplary embodiments, pre-filled disposable pods may be filled with preservative free medication, or virtually any solution configured for delivery to the eye. The pods may be recyclable and can be replaced easily. The use of pre-filled disposable pods prevents contamination. Additionally, pre-filled disposable pods enable users to be able to use a variety of medicines with a single device. Many eye solution delivery systems use reservoirs built into the device which may be unhygienic and cumbersome for the user.

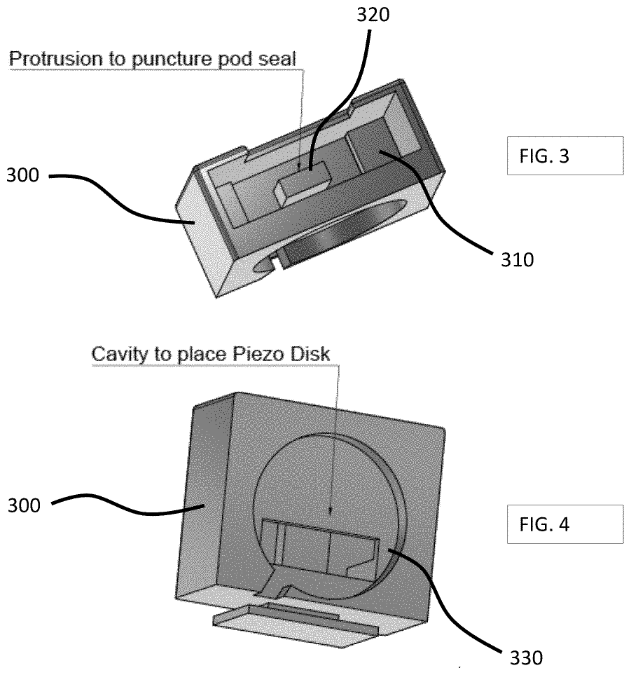

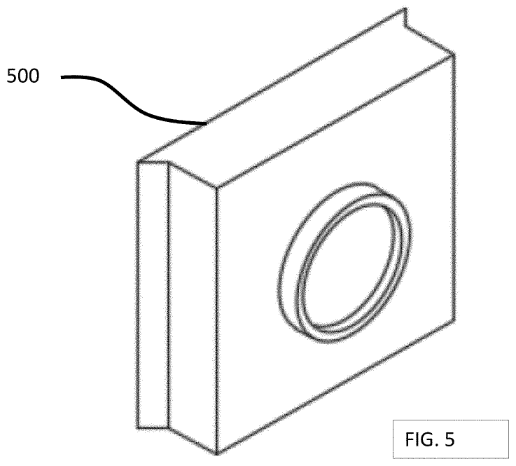

Referring now to FIG. 3, a pod holder 300 is depicted. Pod holder 300 includes a pod cavity 310 for holding a disposable or replaceable pod. Within pod cavity 310 is a protrusion 320 configured to puncture a seal of a pod such as pod 500 of FIG. 5. Pod holder 330 includes a piezo disk cavity 330 which is configured to hold a mesh piezo disk for creating the deliverable mist of the eye solution. In some embodiments pod 500 insertable into pod cavity 310 is disposable and replaceable. In other embodiments, the combination of the pod and a pod holder, such as pod holder 300 which includes the piezo disk, form a combined disposable pod unit which itself may be configured to be disposable.

In accordance with exemplary embodiments, a piezoelectric mesh disk to convert liquid eye drops into a mist is inserted into piezo disk cavity 330. An example piezo disk may be a piezo disk from Steminc of Davenport, Fla., such as but not limited to a 135 kHz disk which delivers 4 um sized drops. This exemplary configuration enables the ability to apply the mist without having a blink reflex reaction. There exist other combinations of frequency and size which can be used as well. For example, a combination of droplets sized between 2 um-10 um and frequency between 100 khz-200 khz maybe also be used.

In accordance with an exemplary embodiment the mist is applied at a comfortable distance (2-4 inch) from each eye. The momentum of the mist is such that 2-4 inches is ideal as the mist if primarily moving linearly. After 4 inches the mist disperses and partially evaporates. The 2-4 inches also prevents the blink reflex reaction of the user.

In an exemplary embodiment, 3 seconds of applying the mist equals to 1 dose of applied medication however any other amounts and times may be equally applicable.

In use, a user will insert the pod in the slot which will rupture a pod seal releasing the contained fluid. The fluid will meet the back plate of the piezo disk. Activation of the disk will result in the fluid getting forced through the tiny openings on the disk. Upon insertion no fluid will leak out. No fluid will leak out even if the device is inverted. A user will be able to remove the pod even after it is punctured to check pod level or to replace with a new pod. The punctured pod will be able to contain the fluid and will not leak once removed from the device. Upon rupture the fluid may meet the piezo disk in any fashion.

Disposable pods, such as pods 500 will be used in two ways: (1) Pods will contain preservative over the counter eye drops. This pod will last the user 1 week; or (2) Pods will contain preservative free over the counter eye drops. This type of pod will be single use only.

In accordance with an exemplary embodiment, the pod/device maybe used for applications in cosmetics, veterinary ophthalmic delivery, eye lid scrubs, etc. The device may contain an Ultraviolet LED to prevent bacteria build up. The device may contain an LED which lights on once the device is within 2-4 inches from the eye. The pods will be manufactured using recyclable materials. Users maybe able to change the velocity of the mist depending on intended comfort.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flows are presented in a sequence(s), it should be understood that the various operations may be performed in other orders than those which are illustrated or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

What is claimed is:

1. An eye misting device, comprising:
a graspable housing;
a mist aperture in the graspable housing;
a pod holder receiver formed integral the housing;
a pod holder configured to engage the pod holder receiver; and
a pod receiving cavity formed within the pod holder for holding an eye solution pod.

2. The device of claim 1, wherein the housing defines a generally elongate rectangular box.

3. The device of claim 1, wherein the eye solution pod is generally rectangular.

4. The device of claim 1, wherein the pod holder is slidably engageable to the housing.

5. The device of claim 1, wherein the pod holder includes a piezo disk cavity.

6. The device of claim 1, wherein the pod holder includes a protrusion configured to puncture a portion of the eye solution pod when the eye solution pod is inserted into the pod cavity.

7. The device of claim 1, wherein the eye solution pod is disposable.

8. The device of claim 1, wherein the eye solution pod is filled with over-the-counter eye drop solution.

9. The device of claim 1, wherein the pod holder includes a piezo disk cavity and a piezo disk.

10. The device of claim 1, wherein the pod holder includes a piezo disk cavity and a piezo disk and the piezo disk is configured to a produce a mist that does not cause inadvertent blinking of the eye during use.

11. The device of claim 1, wherein the pod holder includes a piezo disk cavity and a piezo disk and the piezo disk is configured to a produce a mist that does not cause inadvertent blinking reflex.

12. The device of claim 1, wherein the pod holder and the eye solution pod are formed as one component.

13. The device of claim 1, wherein the pod holder and the eye solution pod are formed as one component and the one component is disposable.

14. The device of claim 1, further comprising: a battery disposed within the housing.

15. The device of claim 1, further comprising: a battery disposed within the housing, wherein the battery is rechargeable.

16. An eye misting device, comprising:
a graspable housing;
a mist aperture in the graspable housing;
a pod holder configured to engage the housing; and
a pod receiving cavity formed within the pod holder for holding an eye solution pod; and
a piezoelectric mesh disk configured to create a mist of eye solution when electrified.

17. The device of claim 16, wherein the pod holder includes a protrusion configured to puncture a portion of the eye solution pod when the eye solution pod is inserted into the pod cavity.

18. The device of claim 16, wherein the eye solution pod is disposable.

19. The device of claim 16, wherein the eye solution pod is filled with over-the-counter eye drop solution.

20. An eye misting device, comprising:
a graspable housing;
a mist aperture in the graspable housing;
a battery within the housing;
a pod holder configured to engage the housing; and
a pod receiving cavity formed within the pod holder for holding an eye solution pod; and
a piezoelectric mesh disk configured to create a mist of eye solution when electrified by the battery.
